(19) United States
(12) Patent Application Publication
(10) Pub. No.: US 2018/0132728 A1
(43) Pub. Date: May 17, 2018

(54) DRY COUPLING APPARATUS FOR PHOTOACOUSTIC COMPUTED TOMOGRAPHY SYSTEMS

(71) Applicant: Washington University, St. Louis, MO (US)

(72) Inventors: Lihong Wang, St. Louis, MO (US); Cheng-hung Yeh, St. Louis, MO (US); Lei Li, St. Louis, MO (US)

(21) Appl. No.: 15/801,748

(22) Filed: Nov. 2, 2017

Related U.S. Application Data
(60) Provisional application No. 62/416,360, filed on Nov. 2, 2016.

(51) Int. Cl. A61B 5/00; A61B 6/03; A61D 7/04

(52) U.S. Cl. CPC A61B 5/0095; A61B 2503/40; A61D 7/04; A61B 6/032

(57) ABSTRACT

An apparatus for holding an imaging subject, such as a whole animal, within a photoacoustic computed tomography system with dry acoustic coupling is disclosed. The apparatus includes a dry acoustic coupler with a tubular elastic membrane made of an optically and acoustically transmissive material. The tubular elastic membrane defines a lumen and at least one lumen opening proximate to a membrane end. In use, the lumen of the tubular elastic membrane is positioned within a coupling fluid contained within a tank of the photoacoustic computed tomography system. The apparatus enables contact-free acoustic coupling of the imaging subject with the coupling fluid during PA imaging. The tank may be optionally pressurized to enhance stabilization of the imaging subject within the coupling fluid.

### CROSS-REFERENCE TO RELATED APPLICATION

This application claims benefit of priority to U.S. Provisional Application 62/416,360, filed on Nov. 2, 2016 and titled “DRY COUPLING FOR PHOTOACOUSTIC COMPUTED TOMOGRAPHY,” which is hereby incorporated by reference in its entirety and for all purposes.

### STATEMENT REGARDING FEDERALLY SPONSORED RESEARCH & DEVELOPMENT

This invention was made with government support under Grant No. U01 NS090579 awarded by National Institutes of Health. The U.S. government has certain rights in the invention.

### BACKGROUND

Aspects of the disclosure relate generally to techniques for coupling a photoacoustic (PA) imaging system to a tissue or animal to be imaged. In particular, aspects of the disclosure relate to techniques for dry coupling a PA imaging system to a whole animal to be imaged in vivo.

Whole-body small-animal imaging is widely used in biomedical research for studying and modeling human disease. However, most whole-body small-animal imaging modalities, such as magnetic resonance imaging (MRI), positron emission tomography (PET), and X-ray computed tomography (CT), have limitations. For example, MRI requires a high-strength magnetic field and long imaging time, and X-ray CT and PET utilize ionizing radiation, which may limit the ability to obtain longitudinal experimental results.

Recently, there has been increased interest in whole-body small-animal photoacoustic tomography (PAT). In PAT, photons are absorbed by biomolecules within tissue, and subsequently the absorbed energy is converted to heat, producing ultrasonic pressure waves via thermoelastic expansion. By detecting these ultrasonic pressure waves, PAT yields high-resolution images in both the ballistic and diffusive optical regimes. Over the past few years, multiple whole-body small-animal PAT systems have been implemented using different acoustic coupling media, light delivery systems, and acoustic detection designs. However, due to limited detection views, hemispherical array photoacoustic computed tomography and half-ring multispectral optoacoustic tomography (MSOT) generally require rotating the animal to achieve full view detection. Moreover, MSOT enabled with fiber bundle illumination lacks uniformity in light delivery, which degrades image quality. Ring-shaped confocal photoacoustic computed tomography (RC-PACT) utilizes both full-ring light illumination and full-ring acoustic detection to address these issues. However, similar to other whole-body small-animal imaging systems, RC-PACT makes use of water as a direct-contact coupling medium, which can induce anxiety and water-immersion wrinkling in mice. Both of these factors can render physiological measurements inaccurate in various ways, such as by decreasing T-cell blastogenesis, altering blood flow velocity, and inducing vasoconstriction.

### SUMMARY

In one aspect, a dry acoustic coupling apparatus for positioning an imaging subject within a photoacoustic imaging system is provided. The apparatus includes a flexible tubular membrane comprising an acoustically and optically transmissive material, and having a first membrane end and a coupling membrane portion coupled to the first membrane end. The apparatus also includes a lumen defined by the flexible tubular membrane and comprising a first lumen opening proximate the first membrane end. The lumen is configured to receive at least a portion of the imaging subject via the first lumen opening. During operation, at least part of the coupling membrane portion is positioned within an acoustic coupling fluid of the photoacoustic imaging system and the flexible tubular membrane is disposed between the at least a portion of the imaging subject within the lumen and the acoustic coupling fluid.

In another aspect, a dry-coupled photoacoustic computed tomography system for obtaining a photoacoustic image of an imaging subject is provided. The system includes a photoacoustic computed tomography system comprising a tank defining a cavity containing an acoustic coupling fluid. The system also includes a dry acoustic coupling apparatus coupled to the photoacoustic computed tomography system. The dry acoustic coupling apparatus comprises a flexible tubular membrane made of an acoustically and optically transmissive material. The flexible tubular membrane includes a first membrane end and a coupling membrane portion coupled to the first membrane end. The flexible tubular membrane defines a lumen with a first lumen opening proximate the first membrane end. The lumen is configured to receive at least a portion of the imaging subject via the first lumen opening. During operation at least part of the coupling membrane portion is positioned within the acoustic coupling fluid in the tank and the flexible tubular membrane is disposed between the at least a portion of the imaging subject within the lumen and the acoustic coupling fluid.

In an additional aspect, a full-ring dry-coupled confocal whole-body photoacoustic computed tomography system configured to obtain a photoacoustic image of an imaging subject is provided. The system includes a tank defining a cavity containing an acoustic coupling fluid. The tank includes a first face defining a first cavity opening of the cavity and a second face defining a second cavity opening of the cavity, in which the second face is opposite the first face. The system also includes a first plate sealed over the first face. The first plate defines a first plate opening passing through the first plate into the cavity. The first comprises an optically transmissive material. The system further includes a second plate sealed over the second face and defining a second plate opening passing through the second plate into the cavity. The system additionally includes a tubular flexible membrane comprising an acoustically and optically transmissive material. The tubular flexible membrane further comprises a coupling membrane portion disposed between a first membrane end and a second membrane end. The tubular flexible membrane defines a lumen comprising a first lumen opening proximate the first membrane end and a second lumen opening proximate the second membrane end. The lumen is configured to receive the imaging subject via the first lumen opening. The first membrane end is coupled around a perimeter of the first plate opening opposite to the cavity of the tank. The second membrane end is coupled around a perimeter of the second plate opening opposite to the cavity of the tank. At least a portion of the coupling membrane portion extends through the acoustic coupling fluid in the tank. The system additionally includes a pulsed laser and associated optics configured to deliver at least one laser pulse into an optical focus region within the cavity of the tank and a ring ultrasound transducer array configured to detect photoacoustic signals produced within an acoustic focus region. At least part of the coupling membrane portion is disposed between at least a portion of the imaging subject within the lumen and the acoustic coupling fluid. The optical focus region and the acoustic focus region coincide at a region of interest positioned within at least a portion of the imaging subject within the lumen and the acoustic coupling fluid. The system is further configured to reconstruct a 2D photoacoustic image of the region of interest based on the photoacoustic signals detected by the ring ultrasound transducer array. The photoacoustic signals are elicited in response to illumination by the at least one laser pulse directed to the optical focus region.

In certain aspects, a dry acoustic coupling apparatus, implemented for animal coupling in a photoacoustic computed tomography system, is configured to substantially decrease water immersion anxiety and/or wrinkling of the subject animal being imaged. In addition or alternatively, the dry acoustic coupling apparatus is configured to facilitate incorporating complementary modalities and procedures.

These and other features and advantages will be described in further detail below, with reference to the associated drawings.

### BRIEF DESCRIPTION OF THE DRAWINGS

FIG. 1 is an exploded-view schematic diagram of a dry coupling device according to one aspect.

FIG. 2 is a schematic diagram of a full-ring dry-coupled confocal whole-body photoacoustic computed tomography (RDC-PACT) system according to one aspect including the dry coupling device shown in FIG. 1.

FIG. 3 is a schematic diagram of an animal holder according to one aspect including the dry coupling device and full-ring dry-coupled confocal whole-body photoacoustic computed tomography (RDC-PACT) system shown in FIG. 1 and FIG. 2, respectively.

FIG. 4A is an in vivo RC-PACT image of the liver region of a nude mouse obtained using a conventional water coupling device.

FIG. 4B is a close-up of the in vivo RC-PACT image of FIG. 4A.

FIG. 4C is an in vivo RC-PACT image of the liver region of a nude mouse obtained using a dry coupling device according to one aspect.

FIG. 4D is a close-up of the in vivo RC-PACT image of FIG. 4C.

FIG. 4E is a graph comparing the contrast-to-noise ratio (CNR) obtained from the image of FIG. 4A (water coupling) and from the image of FIG. 4C (dry coupling).

FIG. 5A is an in vivo RC-PACT image of the kidney region of a nude mouse obtained using a water coupling device.

FIG. 5B is a close-up of the in vivo RC-PACT image of FIG. 5A.

FIG. 5C is an in vivo RC-PACT image of the kidney region of a nude mouse obtained using a dry coupling device in one aspect.

FIG. 5D is a close-up of the in vivo RC-PACT image of FIG. 5C.

FIG. 5E is a graph comparing the contrast-to-noise ratio (CNR) obtained from the image of FIG. 5A (water coupling) and from the image of FIG. 5C (dry coupling).

### DETAILED DESCRIPTION

In various aspects, systems and methods for enhanced photoacoustic computed tomography (PAT) with dry acoustic coupling are disclosed. In certain aspects, a dry acoustic coupling apparatus includes a flexible tubular membrane with a lumen configured to receive a subject to be imaged using PAT within a tank containing an acoustic coupling fluid such as, for example, water. The flexible tubular membrane enables acoustic coupling of the imaging subject with the acoustic coupling fluid of the PAT device without placing the imaging subject in direct contact with the acoustic coupling fluid.

In some aspects, the tank containing the membrane and acoustic coupling fluid is sealed to provide for pressurization (i.e. a pressurized tank) to further enhance stabilization of the imaging subject and to eliminate bubbles within the lumen of the membrane that may introduce artifacts to the PAT images of the imaging subject. The dry acoustic coupling apparatus is compatible with a variety of PAT systems such as, for example, a whole-body small-animal ring-shaped photoacoustic computed tomography system, as described in additional detail below.

The dry acoustic coupling apparatus of certain aspects may overcome one or more limitations of conventional photoacoustic computed tomography (PACT) systems. In certain cases, the flexible tubular membrane (e.g., a tubular membrane made of elastic material(s)) that encloses the subject to be imaged in a PAT system results in a 2D/3D PAT image quality that is level comparable to that of PAT imaging using conventional water coupling typical of conventional PAT imaging systems. In addition or alternatively, the sequestration of the imaging subject from the acoustic coupling fluid of the PAT imaging system within the membrane of the dry acoustic coupling apparatus, may reduce anxiety and/or wrinkling of the imaging subject, for example, where the imaging subject is a small animal to be immersed in a tank containing water as the acoustic coupling fluid. As another advantage, the flexible tubular membrane of the dry acoustic coupling apparatus may also reduce contamination of the acoustic coupling fluid from shedding of tissues or waste products associated with the imaging subject, thereby simplifying maintenance of the PAT system. In addition, the dry coupling apparatus of certain aspects provide for access to the imaging subject while positioned within the PAT system, thus facilitating the incorporation of complementary modalities and procedures, such as, for example, monitoring physiological parameters such as heart and respiration rate, brain activity, injections of compounds into the imaging subject at any time during the PAT imaging procedure, and any combination thereof. Further, because the acoustic coupling fluid does not contact the imaging subject, a wider variety of compositions of acoustic coupling fluid may be selected for use with the PAT imaging system independently of the biocompatibility of the composition.

In various aspects, the dry acoustic coupling apparatus is configured to hold an imaging subject within a photoacoustic imaging system such that the imaging subject is acoustically and optically coupled to the corresponding elements of the photoacoustic imaging system, using a dry coupling method in which the imaging subject is contained within a flexible tubular membrane that enables the imaging subject to be submersed within an acoustic coupling fluid with the imaging subject and the acoustic coupling fluid separated by the flexible tubular membrane. The dry acoustic coupling apparatus is configured to position any suitable imaging subject within a photoacoustic imaging system such as, for example, a portion or a whole body of an animal such as a mouse, or a portion of a body of a pediatric human patient such as an appendage of a human patient, e.g., a finger, toe, cranial portion, arm, leg, or any other suitable appendage or portion thereof

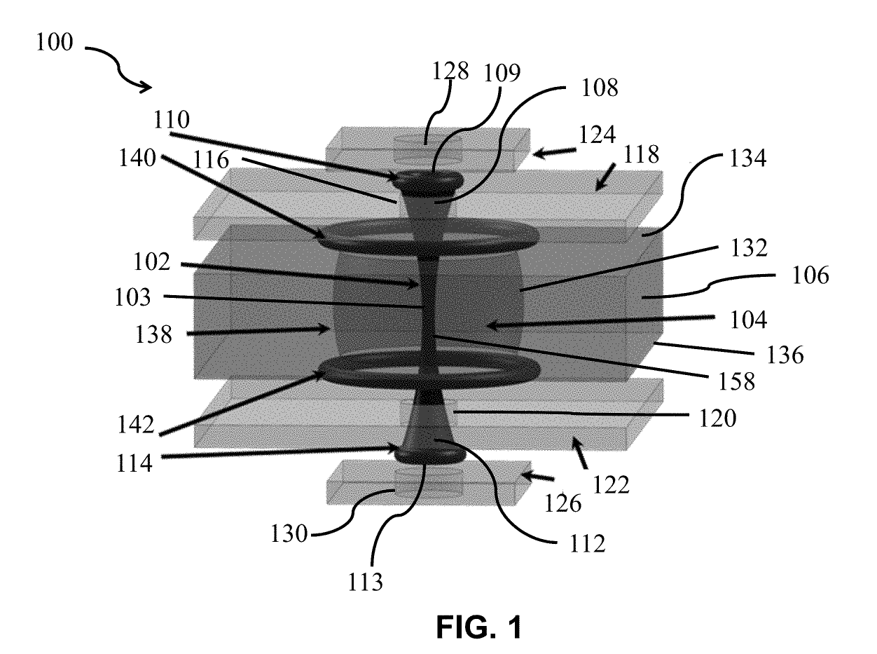

An exploded view schematically illustrating a dry acoustic coupling apparatus 100 of one aspect is provided as FIG. 1. The illustration also includes components of a dry-coupled photoacoustic computed tomography system. The dry acoustic coupling apparatus 100 includes a flexible tubular membrane 102 positioned within a tank 106 containing an acoustic coupling fluid 104 such as, for example, water. The flexible tubular membrane 102 includes a first membrane end 108 and a second membrane end 112 opposite the first membrane end 108. The first membrane end 108 is affixed to a first membrane attachment fitting 110 such as, for example, a first O-ring or other suitable seal. The second membrane end 112 is affixed to a second membrane attachment fitting 114 such as, for example, a second O-ring or other suitable seal. A coupling membrane portion 103 is positioned within the acoustic coupling fluid 104.

In various aspects, the flexible tubular membrane is produced from any biocompatible elastic material that is additionally at least partially optically and acoustically transmissive without limitation. The thin membrane material of the membrane tube minimizes light and sound attenuation between the tank and the imaging subject within the flexible tubular membrane. Non-limiting examples of acoustically and optically transmissive materials suitable for the construction of the tubular flexible membrane include elastic latexes, silicone polymers, polyurethanes, and any other suitable material known in the art. In one aspect, the membrane is constructed from an elastic latex material with a thickness of about 20 μm.

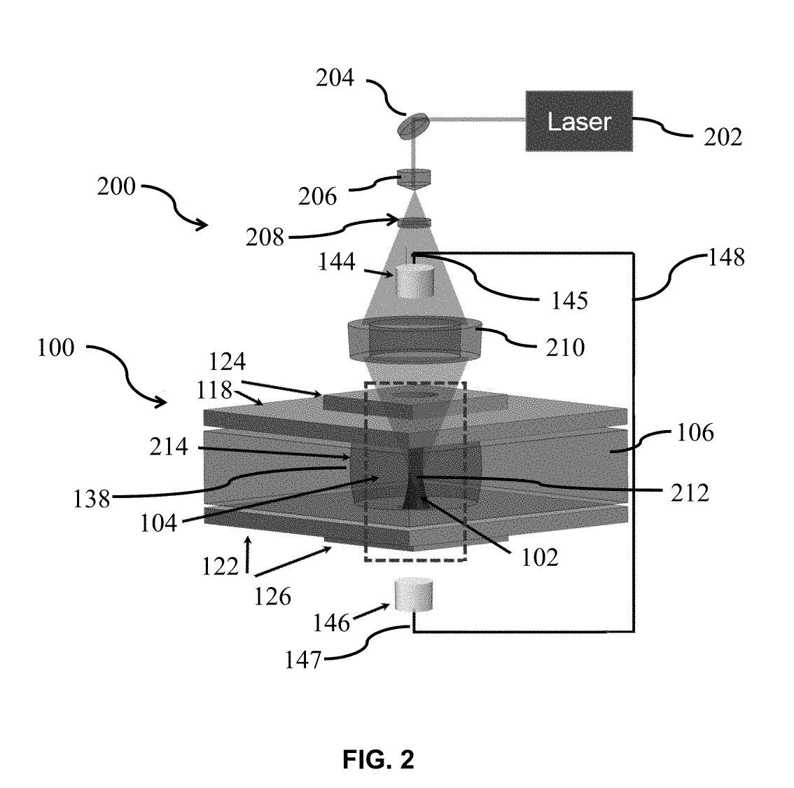

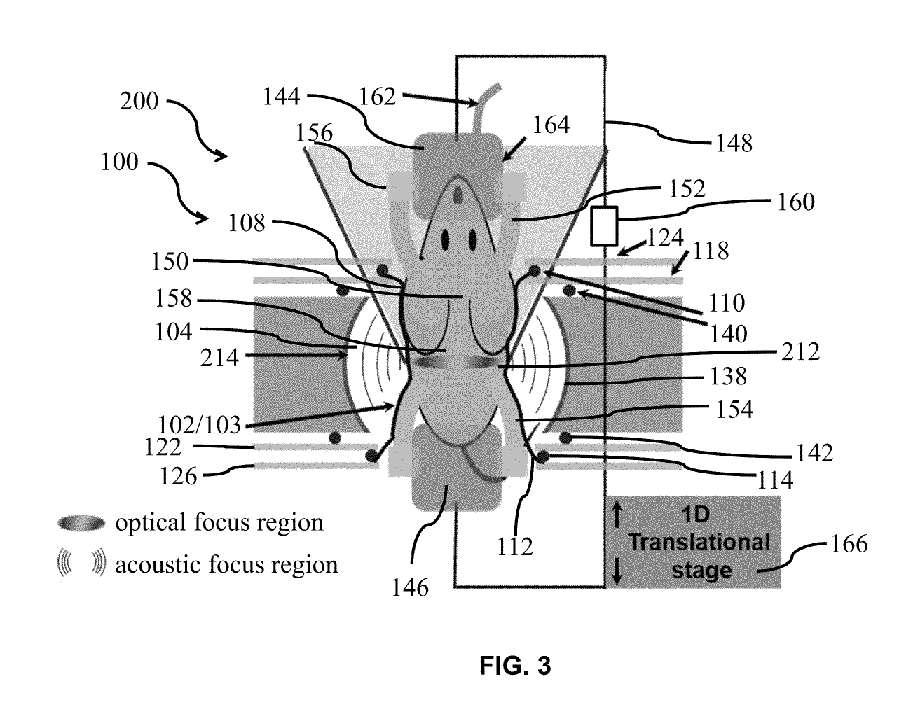

In certain aspects, the dry acoustic coupling apparatus 100 further includes a frame 148 (as shown in FIGS. 2 and 3) having a first mounting base 144 coupled to a first frame end 145 and a second mounting base 146 coupled to a second frame end 147 opposite the first frame end 145. The first mounting base 144 is configured to hold a first portion of the imaging subject 150 protruding from the first lumen opening 109 and the second mounting base 146 is configured to hold a second portion of the imaging subject 150 protruding from the second lumen opening 113 such that the imaging subject 150 within the lumen 158 defined by the interior walls of the flexible tubular membrane 102 is supported between and by the first and second mounting bases 144 and 146.

Returning to FIG. 1, the dry acoustic coupling apparatus 100 further includes a first plate 118 and a second plate 122 positioned on opposite faces of the tank 106. A first plate opening 116 is formed within the first plate 118 and a second plate opening 120 is formed within the second plate 122. The first plate opening 116 is configured to retain the first membrane attachment fitting 110 and the second plate opening 120 is configured to retain the second membrane attachment fitting 114 such that the flexible tubular membrane 102 is positioned within the acoustic coupling fluid 104 within the tank 106. In the illustrated example shown in FIG. 1, the first membrane attachment fitting 110 and second membrane attachment fitting 114 are O-rings with a diameter larger than a corresponding diameter of the first plate opening 116 and the second plate opening 120, respectively. In another aspect, (not illustrated) circular grooves may be formed in the material of the first plate 118 and the second plate 122 surrounding the first plate opening 116 and second plate opening 120, respectively, to receive the first membrane attachment fitting 110 and the second membrane attachment fitting 114.

In another aspect, the dry acoustic coupling apparatus 100 further includes a third plate 124 positioned against the first membrane attachment fitting 110 opposite the first plate 118, as well as a fourth plate 126 positioned against the second membrane attachment fitting 114 opposite the second plate 122. In use, the third plate 124 and the first plate 118 are configured to retain the first membrane attachment fitting 110 when the third plate 124 is compressed toward the first plate 118, compressing the first membrane attachment fitting 110 between the first plate 118 and the third plate 124. Further, the fourth plate 126 and the second plate 122 are configured to retain the second membrane attachment fitting 114 when the fourth plate 126 is compressed toward the second plate 122. In another aspect, a third plate opening 128 is formed within the third plate 124 and a fourth plate opening 130 is formed within the fourth plate 126 to provide access to the inner lumen 158 of the membrane 102 via the first membrane end 108 and second membrane end 112, respectively, of the flexible tubular membrane 102.

In an additional aspect (not illustrated), the third plate 124 and the first plate 118 may further include aligned circular depressions formed within the respective mating surfaces surrounding the third plate opening 128 and the first plate opening 116 to retain the first membrane attachment fitting 110 therebetween. Similarly, the fourth plate 126 and the second plate 122 may further include aligned circular depressions formed within the respective mating surfaces surrounding the fourth plate opening 130 and the second plate opening 120 to retain the second membrane attachment fitting 114 therebetween.

In another aspect, the first plate 118 is sealed against a first face 134 of the tank 106 and the second plate 122 is sealed against a second face 136 of the tank 106 opposite the first face 134 to seal the acoustic coupling fluid 104 within a cavity 138 formed within the tank 106. In this aspect, the dry acoustic coupling apparatus 100 further includes a third O-ring 140 or other suitable seal compressed between the first plate 118 and the first face 134, and a fourth O-ring 142 or other suitable seal compressed between the second plate 122 and the second face 136 to enhance the sealing of the acoustic coupling fluid 104 within the cavity 138. In this aspect, first plate 118, second plate 122, third plate 124, and fourth plate 126 are acrylic plates. In other aspects, first plate 118, second plate 122, third plate 124, and fourth plate 126 can be made of any suitable material for the PA imaging system described below.

In various aspects, the dry acoustic coupling apparatus 100 is compatible for use with a variety of photoacoustic imaging systems without limitation. In these various aspects, the dry acoustic coupling apparatus 100 can be combined with a photoacoustic imaging system to assemble a dry-coupled photoacoustic imaging system such as, for example, a dry-coupled confocal photoacoustic computed tomography system, a dry-coupled whole-body photoacoustic computed tomography system, and a dry-coupled confocal whole-body photoacoustic computed tomography system.

FIG. 2 is a schematic illustration of a full-ring dry-coupled confocal whole-body photoacoustic computed tomography (RDC-PACT) system 200 and the dry acoustic coupling apparatus 100 in one aspect. In this aspect, the RDC-PACT system 200 includes a pulsed laser 202 configured to produce a series of laser pulses and associated optics to direct the series of laser pulses (not illustrated) into the body of the animal (not illustrated) positioned within the flexible tubular membrane 102 at an optical focus region 212. The pulsed laser 202 includes any known and suitable light source without limitation. Non-limiting examples of a suitable light sources include a solid-state laser with a 7 ns pulse duration and 50 Hz pulse repetition rate, such as, for example, the DLS 9050 laser made by Continuum® of San Jose, Calif. and associated optical fibers.

As illustrated in FIG. 2 in one aspect, the optics include a mirror 204, a conical lens 206, an optical diffuser 208, and an optical condenser 210. The mirror 204 is configured to redirect the series of laser pulses produced by the laser 202 into the conical lens 206. The conical lens 206 is configured to form a ring-shaped light beam and to expand the series of laser pulses into the optical diffuser 208. The optical diffuser 208 is configured to homogenize the cross-sectional energy distribution of the series of laser pulses. The optical condenser 210 is configured to refocus the series of laser pulses into an optical focus region 212 within the membrane 102 and provide uniform illumination and full-ring light delivery to imaging subject 150 at the optical focus region 212.

In various aspects, the laser pulses used to elicit photoacoustic (PA) signals from the imaging subject during imaging may be delivered to an optical focus region by any known means without limitation, such as, for example, optical fibers or another suitable mechanism for focusing light in the imaging plane. The illuminated region within an RDC-PACT system may be translated or rotated during use to image the entire region of interest in two dimensions (2D PA imaging) and/or three dimensions (3D PA imaging).

Referring again to FIG. 2, the RDC-PACT system 200 further includes an ultrasound transducer array 214 positioned around a perimeter of the cavity 138. In various aspects, the ultrasound transducer array 214 is any known ultrasound detection device such as, for example, a full-ring transducer array, a half-ring transducer array, or a hemispherical array that may be fixed or rotated. In one aspect, the ultrasound transducer array 214 is positioned and configured such that the optical focus region 212 of the laser pulses are contained within the acoustic focus region 213 of the ultrasound transducer array 214, as discussed in additional detail below.

In various aspects, the choice of the illumination or detection schemes may influence the speed of imaging by RDC-PACT 200. Without being limited to any particular theory, the RDC-PACT system 200 illustrated in FIG. 2, which provides for obtaining PA signals sufficient to reconstruct a complete PA image within the imaging plane from a single light pulse, may provide for a higher imaging speed relative to alternative systems that may require multiple light pulses to acquire PA signals sufficient to reconstruct a complete PA image within the imaging plane, for example, due to incomplete coverage of the imaging plane by the light delivery system and/or the ultrasound detection device. By way of a non-limiting example, conventional half-ring and hemispherical transducer array-based photoacoustic tomography systems are associated with limited detection views and image reconstruction artifacts. In addition, a half-ring based system may envelop the whole imaging subject (e.g., small animal) in the flexible tubular membrane, rather than just the portion of the imaging subject to be imaged at any time. The RDC-PACT system 200 illustrated in FIG. 2 contacts only the region being imaged with the flexible tubular membrane and coupling gel.

FIG. 3 is a cross-sectional schematic view of an imaging subject 150 held within the dry acoustic coupling apparatus 100 within the RDC-PACT system 200 illustrated in FIG. 2. In use, the imaging subject 150 is positioned within a lumen 158 defined by the interior walls of the flexible tubular membrane 102 such that at least part of the coupling membrane portion 103 of the flexible tubular membrane 102 is disposed between the portion of the imaging subject 150 within the lumen 158 and the acoustic coupling fluid 104. A first portion 152 of the imaging subject 150 is coupled or otherwise held to the first mounting base 144 and a second portion 154 of the imaging subject 150 is coupled or otherwise held to the second mounting base 146 using one or more fasteners 156. Any known suitable fastener may be used to attach the first portion 152 and second portion 154 of the imaging subject 150 to the first mounting base 144 and second mounting base 146, respectively, without limitation. Non-limiting examples of suitable fasteners 156 include: Velcro fasteners, tapes including surgical tape, elastic bands, hydrogels, padded clamps, and glues including surgical glue.

In another aspect, the dry acoustic coupling apparatus 100 further includes a frame 148 configured to maintain the first mounting base 144 and second mounting base 146 at a constant separation distance. In one example, the first mounting base 144 and the second mounting base 146 are aluminum tubes fastened to the frame 148. The frame 148 includes any known and suitable rigid structural element without limitation. Non-limiting examples of suitable frame structures include a C-shaped metal rod, as illustrated in FIG. 2 and FIG. 3, a C-shaped glass rod, and a curved metal rod. In an additional aspect, the frame 148 is further provided with a separation distance adjustment element 160 configured to adjustably alter the separation distance between the first mounting base 144 and the second mounting base 146 to compensate for various factors such as, for example, variations in animal morphology including length and height of the animal, and to position the animal in a posture favorable for imaging by the photoacoustic imaging system. Non-limiting examples of devices suitable for use as separation distance adjustment element 160 include linear actuators such as screwjacks, rack and pinions, pneumatic actuators, roller screws, and hoists.

By way of non-limiting example, the separation distance between the first mounting base 144 and second mounting base 146 are increased after attaching the first portion 152 and second portion 154 of the animal, respectively, to ensure that the imaging subject 150 is positioned in a fully extended posture to reduce movements of the imaging subject 150 during imaging.

In another aspect, the dry acoustic coupling apparatus 100 is further provided with a gas supply tube 162 coupled to the first mounting base 144 at a first tube end. The gas supply tube 162 is configured to provide gases such as, for example, oxygen, gaseous anesthesia compounds, and any combination of gaseous compounds thereof to the imaging subject 150 within the flexible tubular membrane 102. A source of the gas compound (not illustrated) is provided to the gas supply tube 162 at a second tube end (not illustrated). In an additional aspect, the gas supply tube 162 may be operatively connected to a gas mask or any other known means of delivering gases to the imaging subject 150 including, but limited to, a gas mask. In another additional aspect, a gas delivery recess 164 (e.g., in the form of a gas mask) is formed into the first mounting base 144. In this aspect, the gas delivery recess 164 is configured to deliver gases to the imaging subject 150. The gas delivery recess 164 is operatively coupled to the gas supply tube 162 at a first tube end to ensure adequate gas supply to the imaging subject 150 via the gas delivery recess 164.

In this aspect, the imaging subject 150 is inserted into the lumen 158 of the flexible tubular membrane 102 and coupled to the first mounting base 144 and the second mounting base 146 using the fasteners 156, as shown in FIG. 3, to image the imaging subject 150 in the RDC-PACT system 200, shown in FIG. 2. Inserting the imaging subject 150 into the lumen 158 of the flexible tubular membrane 102 prevents direct contact with the acoustic coupling fluid that may wrinkle or create stress for the imaging subject 150. The RDC-PACT system 200 is considered to be dry coupled because of the lack of direct water contact. In other aspects, the system 200 can be converted into a water-coupled system by removing the flexible tubular membrane 102.

In one aspect, the exterior of the imaging subject 150 may be treated with a known and suitable low acoustic impedance substance (not illustrated) before inserting the imaging subject 150 into the lumen 158 of the flexible tubular membrane 102. Non-limiting low acoustic impedance substances suitable for application to the imaging subject 150 include ultrasound coupling gels. In one aspect, the distance between the first mounting base 144 and the second mounting base 146 is adjusted using the separation distance adjustment element 160 to minimize movements of the imaging subject 150 within the RDC-PACT system 200. In additional or alternatively, the acoustic coupling fluid 104 may be pressurized to further minimize imaging subject 150 movements. The imaging subject 150 is positioned within the lumen 158 of the flexible tubular membrane 102 such that the region of interest to be imaged is aligned with the optical focus region 212 of the laser pulses and within the acoustic focus region 213 of the ultrasound transducer array 214.

In some aspects, the gel layer between the imaging subject 150 and the inner surface of the flexible tubular membrane 102 may potentially contain bubbles, which may distort and reflect ultrasound signals, causing reconstruction artifacts. To mitigate these potential artifacts, the tank 106 may provide for a closed water tank design as described above to enable pressurization of the water or other acoustic coupling fluid 104 within the tank 106, which minimizes any bubbles forming gaps between the imaging subject 150 and the flexible tubular membrane 102, to enhance the acoustic coupling of the imaging subject 150 and the acoustic coupling fluid 104. This pressurization also stabilizes the imaging region without stressing the entire animal. By comparison, fiberglass rods or thin wires in tension attached to the animal are typically used to stabilize the imaging region in conventional water-coupled PAT systems that may induce stress in the animal to be imaged.

By way of a non-limiting example, the dry acoustic coupling apparatus 100 includes the first and second mounting bases 144,146 constructed from aluminum tubes, and the first mounting base 144 is connected to the gas supply tube 162 in an arrangement similar to the dry acoustic coupling apparatus 100 illustrated in FIG. 3. The aluminum tubes are fastened to a frame 148 formed from fixed metal rods (e.g., Mini-series mounting posts, Thorlabs, Inc., Newton, N.J., USA) and moved by a one-dimensional linear stage 166 (PROMECH® LP28 Miniature Linear Positioner, Parker, Charlotte, N.C., USA). The imaging subject 150, such as the mouse illustrated in FIG. 3, is secured by taping its fore and hind legs to the aluminum tubes. Ultrasound gel is then applied to the animal's skin. The rigid design of the metal rods and aluminum tubes minimizes holder movement caused by animal motion such as respiration.

FIG. 3 shows the imaging subject 150 positioned with its abdomen region aligned with the optical focus region 212 of laser pulses and aligned with the acoustic focus of the ultrasound transducer array 214, thereby defining the abdomen region as the region of interest to be imaged. In this example, the imaging subject 150 is positioned vertically within the RDC-PACT system 200. In another aspect, the imaging subject 150 is rotated 90° to mount the imaging subject 150 horizontally within the RDC-PACT system 200, similar to a mounting device of a CT or MRI scanner. The optical focus region 212 of the laser pulses illuminate the tissues of the imaging subject 150 within the region of interest and at least a portion of the laser pulse energy is absorbed and released as ultrasonic and photoacoustic waves within the tissue of the imaging subject 150. The generated photoacoustic waves are detected by the ultrasound transducer array 214. In one aspect, the ultrasound transducer array 214 includes 512 transducer elements arranged in a ring array for data acquisition at a relatively high spatial resolution. The flexible tubular membrane 102 is configured to minimize light and sound attenuation between the acoustic coupling fluid 104 and the imaging subject 150 within the flexible tubular membrane 102. In one aspect, raw data from each transducer element within the ultrasound transducer array 214 is detected in parallel using a 512-channel data acquisition system (not illustrated) to enhance data acquisition speed and signal-to-noise ratio.

Further, in one aspect, PAT images are reconstructed using a universal back-projection algorithm that is well known in the art of imaging. In this aspect, the RDC-PACT system 200 may provide 0.1 mm in-plane resolution and 1 mm elevational resolution.

By way of a non-limiting example, a ring-shaped dry-coupled confocal photoacoustic computed tomography system (RDC-PACT) similar to the system 200 illustrated in FIG. 2 includes a tubular elastic latex membrane animal holder to achieve dry coupling and employs free-space full-ring light delivery to provide high fluence and uniform illumination. This exemplary system uses a solid-state laser (DLS 9050, Continuum) with a 7 ns pulse duration and 50 Hz pulse repetition rate. The laser beam passes through a conical lens (cone angle 130° , Delmar Photonics) to form a ring-shaped light beam and is homogenized by an optical diffuser (EDC-5, RPC Photonics). The beam is focused by an acrylic condenser to achieve full-ring light delivery. The maximum light intensity at the surface of the imaging subject is approximately 20 mJ/cm² at 1064 nm, which is below the safety limit set by the American National Standards Institute (ANSI). The generated photoacoustic waves are detected by a custom-made 512-element full-ring ultrasonic transducer array with a 5 MHz central frequency (>84% one-way detection bandwidth, Imasonic SAS). Compared to conventional RC-PACT systems, the exemplary RDC-PACT system employs a larger full-ring ultrasonic transducer array (10 cm vs. 5 cm diameter), which can accommodate larger animals. The RDC-PACT system provides for 0.1 mm in-plane resolution and 1 mm elevational resolution. Raw channel data from each element are detected in parallel using a 512-channel data acquisition system. Images are reconstructed using the universal back-projection algorithm.

In another aspect, the imaging subject 150 is translated in a linear path along an axis perpendicular to optical focus region 212 of laser pulses to obtain multiple 2D image slices that may be combined using known methods to obtain a 3D image of at least a portion of the imaging subject 150. In one aspect, the dry acoustic coupling apparatus 100 is further provided with an actuated means of linearly translating the imaging subject 150 such as, for example, the 1D linear stage 166 coupled to the frame 148, as illustrated in FIG. 3. Other suitable photoacoustic imaging systems and methods of photoacoustic images are described in additional detail in U.S. Pat. Nos. 8,997,572, 9,226,666, and 9,439,571, the entire contents of which are incorporated by reference herein.

In this aspect, because the RDC-PACT system 200 only contacts the region of interest to be imaged in the imaging subject 150 without submersion within and in direct contact with an acoustic coupling fluid, the RDC-PACT system 200 facilitates access to the other body regions of the imaging subject 150 after the imaging subject 150 is positioned within the flexible tubular membrane 102 inside the tank 106 containing acoustic coupling fluid 104 in preparation for PAT imaging. Access to these other body regions of the imaging subject 150 provides for additional sensors to be attached to the imaging subject 150 such as, for example, EEG leads to monitor brain activity of the animal, ECG leads to monitor heart rate of the imaging subject 150 and/or impedance pneumography leads to monitor respiration of imaging subject 150. Because most of the body regions of the imaging subject 150 are accessible while the imaging subject 150 is positioned in the RDC-PACT system 200, intraperitoneal injections or vein injections at the first portion 152, the second portion 154, and the tail (not illustrated) of the imaging subject 150 may be readily performed.

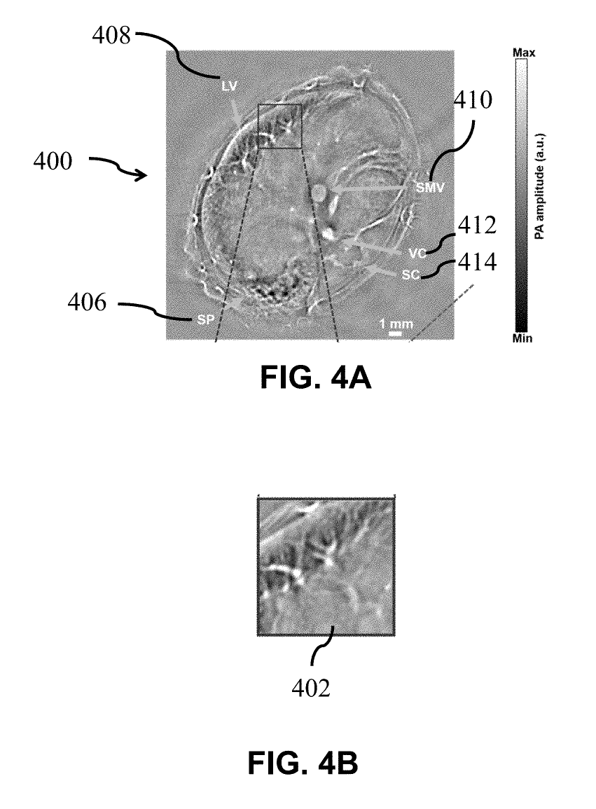

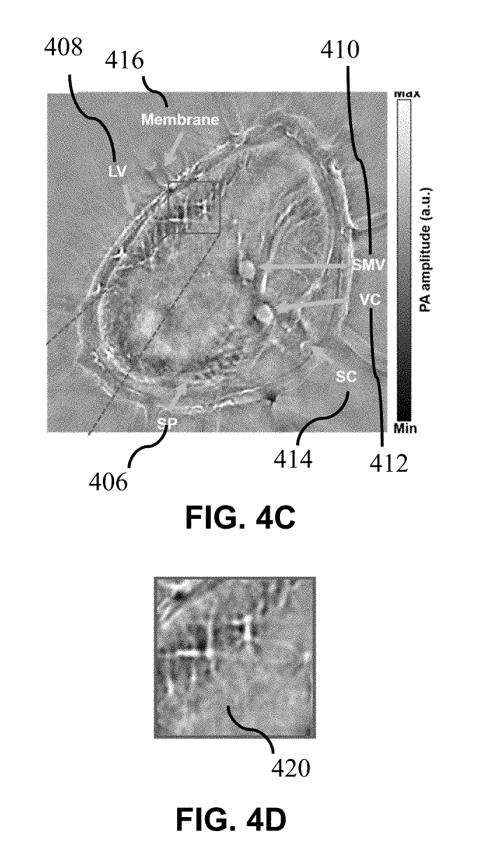

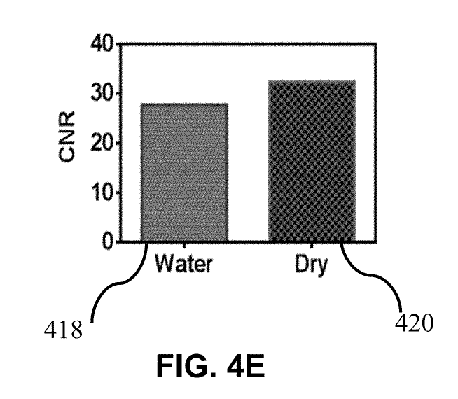

FIG. 4A and FIG. 4B show in-vivo images of the liver region of the animal 150 obtained from a water-coupled photoacoustic (PA) imaging system. FIG. 4C and FIG. 4D show in-vivo images of the liver region of the animal 150 obtained from an RDC-PACT system 200 similar to the system 200 illustrated in FIG. 2. Spleen (SP) 406, liver (LV) 408, superior mesentery vein (SMV) 410, vena cava (VC) 412, spinal cord (SC) 414, and flexible tubular membrane 416 are identified by arrows overlaid on FIG. 4A and FIG. 4C. Zoomed in image 402 of water-coupled PA system (see FIG. 4B) and zoomed in image 404 of RDC-PACT system 200 (see FIG. 4D) show that the images produced from water-coupled PA systems and RDC-PACT system 200 are similar in image quality. FIG. 4E is a graph of contrast to noise ratios (CNR) for zoomed in image 402 and zoomed in image 404 to quantitatively compare the images. Higher CNR values are indicative of enhanced image quality. The water-coupled PA system has a CNR 418 of about 27, and the RDC-PACT system 200 has a CNR 420 of about 31.

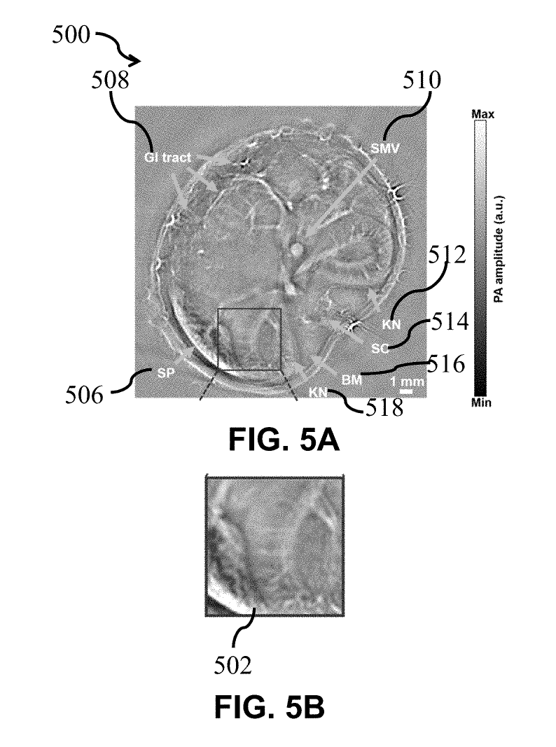

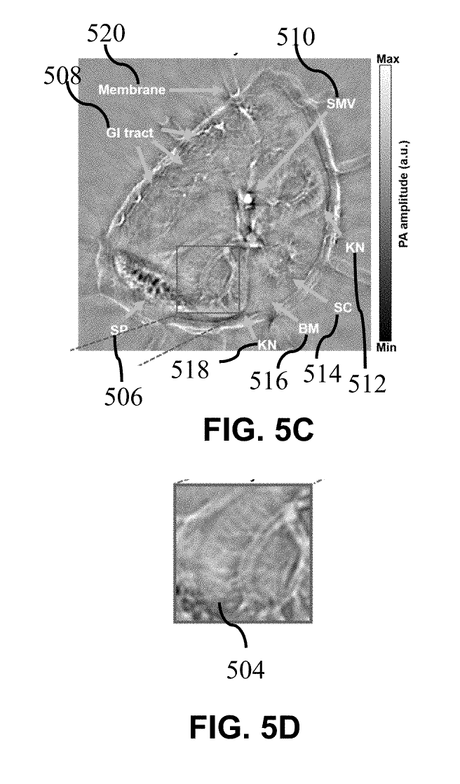

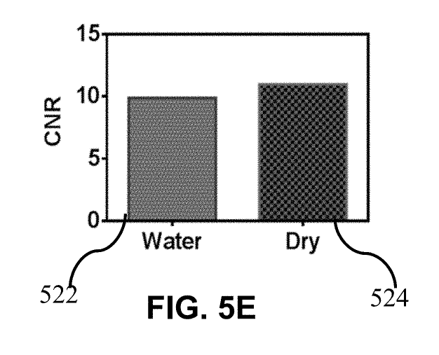

FIG. 5A and FIG. 5B show in-vivo images of the kidney region of the animal 150 obtained from a water-coupled photoacoustic (PA) imaging system. FIG. 5C and FIG. 5D show in-vivo images of the kidney region of the animal 150 obtained from an RDC-PACT system 200 similar to the system 200 illustrated in FIG. 2. Spleen (SP) 506, GI tract 508, superior mesentery vein (SMV) 510, kidneys (KN) 512 and 518, spinal cord (SC) 514, backbone muscle (BM) 516, and flexible tubular membrane 520 are identified by arrows overlaid on FIG. 5A and FIG. 5C. Zoomed in image 502 obtained using the water-coupled PA system (see FIG. 5B) and zoomed in image 504 obtained using RDC-PACT system 200 (see FIG. 5D) show that the images produced from the water-coupled PA system and the RDC-PACT system 200 are similar in image quality. FIG. 5E is a graph of contrast to noise ratios (CNR) for the zoomed in image 502 and the zoomed in image 504 to quantitatively compare the images. Higher CNR values are indicative of enhanced image quality. The water-coupled PA system has a CNR 522 of about 9, and the RDC-PACT system 200 has a CNR 524 of about 12.

FIG. 4E and FIG. 5E show how CNR 420 and CNR 524, from the images obtained using the RDC-PACT system 200, are slightly better than CNR 418 and CNR 522, from the images obtained using the water-coupled PA imaging system. As shown in FIG. 4C and FIG. 5C, RDC-PACT system 200 eliminates water-induced stress and wrinkling of the imaging subject 150, which produces better images. Systems like the RDC-PACT system 200 are less stressful for the imaging subject 150 (e.g., a small animal such as a mouse) during PAT imaging compared to typical water-coupled PA imaging systems in which the animal 150 is typically submerged directly in the acoustic coupling fluid, and these systems are easier to maintain due to isolation of the imaging subject 150 and associated fur and waste products from the acoustic coupling fluid 104. Further, the isolation of the imaging subject 150 from the acoustic coupling fluid enables the selection of the composition of the acoustic coupling fluid without the constraint of biocompatibility with the imaging subject 150 due to the separation of the imaging subject 150 from the acoustic coupling fluid 104 by the physical barrier of the flexible tubular membrane 102. Thus, the composition of the acoustic coupling fluid may include compositions different from deionized water. Some examples of suitable compositions of the acoustic coupling fluid include, but are not limited to, aqueous solutions of varying concentrations including saline solutions, KOH solutions, mineral oils, and any other known acoustic coupling fluids impedance-matched to the body of the imaging subject 150 according to methods well-known in the art.

It should be appreciated by one of ordinary skill in the art that the flexible tubular membrane 102 embodiment of the dry coupling system disclosed herein represents only one possible arrangement. By way of non-limiting example, if one were to extend the technique to certain human applications, such as brain imaging, the dry coupling system of the RDC-PACT system 200 may be modified to create a hemispherical imaging chamber. In this modified system, the ultrasound transducer array 214 would still be situated inside a pressurized tank 106 where coupling between the pressurized tank 106 and the imaging subject 150 occurs via a thin flexible tubular membrane 102 constructed from an acoustically and optically transmissive material. In various aspects, the RDC-PACT system 200 illustrated in FIG. 1, FIG. 2, and FIG. 3 would be immediately applicable to human applications such as imaging a finger without significant modification. The RDC-PACT system 200, with appropriate modification, may be potentially enable additional applications of PACT such as neonatal brain imaging, neonatal body imaging, breast imaging, and extremity imaging.

### EXAMPLES

The following examples demonstrate various aspects of the disclosure.

### Example 1: Comparison of Water Coupling and Dry Coupling in PA Imaging

To experimentally compare whole animal PA imaging using traditional water coupling and dry coupling to hold an animal in position in the RDC-PACT system 200, the imaging subject 150 in the form of the mouse shown in FIG. 3 was imaged using the RDC-PACT system 200 illustrated schematically in FIG. 2 using the dry acoustic coupling device 100 illustrated in FIG. 1.

Water-coupled images of the liver and kidney regions of the mouse, shown in FIG. 4A and FIG. 5A, respectively, were obtained by mounting the mouse in a dry coupling device similar to the apparatus illustrated in FIG. 1 that was reconfigured to enable water coupling by removing the flexible tubular membrane 102 and sealing the bottom of the apparatus with a flexible membrane. Corresponding dry-coupled images, shown in FIG. 4C and FIG. 5C, were obtained by mounting the mouse in the dry acoustic coupling device 100 shown in FIG. 1. Both water-coupled and dry-coupled images were acquired using a laser pulse wavelength of 1064 nm, and endogenous hemoglobin served as the main contrast at this wavelength. All images clearly showed the liver, spleen, and kidneys. Moreover, the entire vascular structure within each of these organs was visible, including the renal artery and venous loop. Further, the spinal cord, backbone muscle, gastrointestinal (GI) tract, superior mesentery vein, and vena cava were clearly imaged.

To examine whether dry coupling could achieve the same image quality as traditional water coupling in PA imaging, the image quality of water-coupled and dry-coupled PA images was compared. To better compare the image quality, the contrast-to-noise ratios (CNR) from several features in the liver region of the mouse (FIG. 4A and FIG. 4C) were plotted, with close-up images (see images of FIG. 4B and FIG. 4D) highlighting one typical feature. The CNRs obtained from the water-coupled image (FIG. 4A) and the dry-coupled image (FIG. 4C) were similar in the overall liver region, as summarized in the graph of FIG. 4E.

The image quality of the water-coupled image (see FIG. 5A) and the dry-coupled image (see FIG. 5C) obtained within the kidney region of the mouse were similarly compared. The kidney region included more organs compared to the liver region previously described above. The CNRs obtained from the water-coupled image (FIG. 5A) and the dry-coupled image (FIG. 5C) were similar in quality throughout the overall kidney region, as summarized in the graph of FIG. 5E. The similar CNR values of FIG. 5E demonstrated that dry coupling can maintain the image quality, even for images of relatively complex tissues, such as the tissues within the kidney region. The comparable CNRs of the water-coupled and dry-coupled images also indicated that the pressurization of the water tank included in the dry coupling system successfully removed bubbles in the gel layer between the animal and the flexible tubular membrane.

### Example 2: Comparison of Water Coupling and Dry Coupling in PA CT Imaging

To demonstrate the capability of continuous whole-body small-animal dry-coupled scanning, a mouse was imaged over a 26-mm length (from the kidney region to the heart region) using a RDC-PACT system similar to the system described in Example 1. A series of dry-coupled in vivo images were obtained over the 26-mm length using the dry coupling devices and methods described in Example 1. A corresponding series of water-coupled images were obtained over a comparable 26-mm length using the water coupling devices and methods described in Example 1. A comparison of the dry-coupled and water-coupled images demonstrated that when pressurization was applied to the water tank of the RDC-PACT system, dry-coupled scanning caused less movement during elevational scanning.

In comparison with earlier whole-body small-animal photoacoustic tomography systems, the specific design of the water tank eliminated water-induced stress and wrinkling. Moreover, the RDC-PACT system, which further incorporated confocal full-ring light delivery, enabled fast full-view cross sectional imaging.

When introducing elements of aspects of the invention or embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Although the foregoing embodiments have been described in some detail to facilitate understanding, the described embodiments are to be considered illustrative and not limiting. It will be apparent to one of ordinary skill in the art that certain changes and modifications can be practiced within the scope of the appended claims. For example, although the illustrated example in FIG. 1 shows a third plate 124 and the fourth plate 126, it would be understood that in other aspects the dry acoustic coupling apparatus 100 omits one or both of the third plate 124 and the fourth plate 126.

In view of the above, it will be seen that the several advantages of the disclosure are achieved and other advantageous results attained. As various changes could be made in the above methods and systems without departing from the scope of the disclosure, it is intended that all matter contained in the above description and shown in the accompanying drawings shall be interpreted as illustrative and not in a limiting sense.

What is claimed is:

1. A dry acoustic coupling apparatus for positioning an imaging subject within a photoacoustic imaging system, the apparatus comprising:
a flexible tubular membrane comprising an acoustically and optically transmissive material, the flexible tubular membrane having a first membrane end and a coupling membrane portion coupled to the first membrane end; and
a lumen defined by the flexible tubular membrane, wherein the lumen comprises a first lumen opening proximate the first membrane end, the lumen configured to receive at least a portion of the imaging subject via the first lumen opening;
wherein during operation at least part of the coupling membrane portion is positioned within an acoustic coupling fluid of the photoacoustic imaging system and the flexible tubular membrane is disposed between the at least a portion of the imaging subject within the lumen and the acoustic coupling fluid.

2. The dry acoustic coupling apparatus according to claim 1, wherein the flexible tubular membrane further having a second membrane end coupled to the coupling membrane portion and opposite the first membrane end, and the lumen further comprises a second lumen opening proximate the second membrane end.

3. The dry acoustic coupling apparatus according to claim 2, further comprising a frame comprising:
a first mounting base coupled to a first frame end; and
a second mounting base coupled to a second frame end, the second frame end opposite the first frame end,
wherein the first mounting base is configured to hold a first portion of the imaging subject protruding from the first lumen opening and the second mounting base is configured to hold a second portion of the imaging subject protruding from the second lumen opening, and wherein the at least a portion of the imaging subject within the lumen is supported between and by the first and second mounting bases.

4. The dry acoustic coupling apparatus according to claim 3, further comprising a linear stage coupled to the frame, the linear stage configured to translate the imaging subject along a linear path within the lumen between the first and second lumen openings.

5. The dry acoustic coupling apparatus according to claim 3, further comprising a gas supply tube coupled to the first mounting base at a first tube end, the gas supply tube configured to deliver a gas compound to the imaging subject.

6. The dry acoustic coupling apparatus according to claim 5, wherein the first mounting base further comprises a gas mask formed into the first mounting base and coupled to the gas supply tube, wherein the gas mask is configured to seal over a portion of the imaging subject and deliver the gas compound to the imaging subject during operation.

7. The dry acoustic coupling apparatus according to claim 1, wherein the at least a portion of the imaging subject within the lumen is positioned within an acoustic focus region of a transducer array of the photoacoustic imaging system and is further positioned within an optical focus region of a light source of the photoacoustic imaging system.

8. A dry-coupled photoacoustic computed tomography system for obtaining a photoacoustic image of an imaging subject, the system comprising:
a photoacoustic computed tomography system comprising a tank defining a cavity containing an acoustic coupling fluid; and
a dry acoustic coupling apparatus coupled to the photoacoustic computed tomography system, the dry acoustic coupling apparatus comprising a flexible tubular membrane made of an acoustically and optically transmissive material, the flexible tubular membrane having a first membrane end and a coupling membrane portion coupled to the first membrane end, the flexible tubular membrane defining a lumen with a first lumen opening proximate the first membrane end, the lumen configured to receive at least a portion of the imaging subject via the first lumen opening,
wherein during operation, at least part of the coupling membrane portion is positioned within the acoustic coupling fluid in the tank and the flexible tubular membrane is disposed between the at least a portion of the imaging subject within the lumen and the acoustic coupling fluid.

9. The dry-coupled photoacoustic computed tomography system according to claim 8, wherein:
the tank further comprises a first face defining a first cavity opening of the cavity; and
the dry acoustic coupling apparatus further comprises a first plate sealed over the first cavity opening, the first plate further comprises a first plate opening, and the flexible tubular membrane further comprises a first membrane attachment fitting coupled to the first membrane end, the first membrane attachment fitting further coupled to the first plate opposite to the cavity of the tank, wherein the coupling membrane portion extends from first membrane end coupled to the first membrane attachment fitting into the acoustic coupling fluid and the first membrane attachment fitting opens into the lumen of the flexible tubular membrane via the first lumen opening.

10. The dry-coupled photoacoustic computed tomography system according to claim 9, further comprising a second membrane end coupled to the coupling membrane portion opposite the first membrane end, the lumen further comprising a second lumen opening proximate the second membrane end, the coupling membrane portion between the first and second membrane ends is positioned within the acoustic coupling fluid.

11. The dry-coupled photoacoustic computed tomography system according to claim 10, wherein:
the tank further comprises a second face opposite the first face, the second face defining a second cavity opening of the cavity; and
the dry acoustic coupling apparatus further comprises a second plate sealed over the second cavity opening, the second plate further comprising a second plate opening and the flexible tubular membrane further comprising a second membrane attachment fitting coupled to the second membrane end, the second membrane attachment fitting further coupled to the second plate opposite to the cavity of the tank, wherein the coupling membrane portion extends between the first and second membrane attachment fittings through the acoustic coupling fluid within the cavity and the second membrane attachment fitting opens into the lumen of the flexible tubular membrane via the second membrane end.

12. The dry-coupled photoacoustic computed tomography system according to claim 8, wherein the tank is a pressurized tank configured to compress the acoustic coupling fluid against the coupling membrane portion and a portion of the at least a portion of the imaging subject within the lumen.

13. The dry-coupled photoacoustic computed tomography system according to claim 11, wherein the dry acoustic coupling apparatus further comprises a frame, the frame comprising:
a first mounting base coupled to a first frame end; and
a second mounting base coupled to a second frame end, the second frame end opposite the first frame end;
wherein the first mounting base is configured to hold a first portion of the imaging subject protruding from the first membrane end and the second mounting base is configured to hold a second portion of the imaging subject protruding from the second membrane end, and wherein the at least a portion of the imaging subject within the lumen is supported between and by the first and second mounting bases.

14. The dry acoustic coupling apparatus according to claim 13, further comprising a linear stage coupled to the frame, the linear stage configured to translate the imaging subject along a linear path within the lumen between the first and second lumen openings to reposition the imaging subject within the lumen.

15. The dry-coupled photoacoustic computed tomography system according to claim 14, wherein the photoacoustic computed tomography system further comprises a transducer array, the transducer array configured to detect a plurality of photoacoustic signals produced by a region of interest, the region of interest comprising a portion of the imaging subject within the lumen positioned within an acoustic focus region of the transducer array.

16. The dry-coupled photoacoustic computed tomography system according to claim 15, wherein the photoacoustic computed tomography system further comprises a light source and optics configured to direct a series of light pulses from the light source into at least the portion of the imaging subject within the lumen positioned within the acoustic focus region.

17. The dry-coupled photoacoustic computed tomography system according to claim 16, wherein the light source and the optics are further configured to direct the series of light pulses into an optical focus region, wherein the optical focus region is contained within the acoustic focus region.

18. The dry-coupled photoacoustic computed tomography system according to claim 17, wherein the first plate comprises an optically transmissive material.

19. A full-ring dry-coupled confocal whole-body photoacoustic computed tomography system configured to obtain a photoacoustic image of an imaging subject, the system comprising:
a tank defining a cavity containing an acoustic coupling fluid, the tank comprising a first face defining a first cavity opening of the cavity and a second face defining a second cavity opening of the cavity, the second face opposite the first face;
a first plate sealed over the first face, the first plate defining a first plate opening passing through the first plate into the cavity, the first plate comprising an optically transmissive material;
a second plate sealed over the second face, the second plate defining a second plate opening passing through the second plate into the cavity;
a tubular flexible membrane comprising an acoustically and optically transmissive material, the tubular flexible membrane further comprising a coupling membrane portion disposed between a first membrane end and a second membrane end, the tubular flexible membrane defining a lumen, the lumen comprising a first lumen opening proximate the first membrane end and a second lumen opening proximate the second membrane end, the lumen configured to receive the imaging subject via the first lumen opening, wherein the first membrane end is coupled around a perimeter of the first plate opening opposite to the cavity of the tank, the second membrane end is coupled around a perimeter of the second plate opening opposite to the cavity of the tank, and at least a portion of the coupling membrane portion extends through the acoustic coupling fluid in the tank;
a pulsed laser and associated optics configured to deliver at least one laser pulse into an optical focus region within the cavity of the tank; and
a ring ultrasound transducer array configured to detect photoacoustic signals produced within an acoustic focus region;
wherein:
at least part of the coupling membrane portion is disposed between at least a portion of the imaging subject within the lumen and the acoustic coupling fluid;
the optical focus region and the acoustic focus region coincide at a region of interest positioned within the at least a portion of the imaging subject within the lumen and the acoustic coupling fluid; and
the system is further configured to reconstruct a 2D photoacoustic image of the region of interest based on the photoacoustic signals detected by the ring ultrasound transducer array, the photoacoustic signals elicited in response to illumination by the at least one laser pulse directed to the optical focus region.

20. The full-ring dry-coupled confocal whole-body photoacoustic computed tomography system according to claim 19, further comprising:
a frame ending at a first mounting base coupled to a first frame end and further ending at a second mounting base coupled to a second frame end, the second frame end opposite the first frame end, the first mounting base configured to hold a first portion of the imaging subject protruding from the first lumen opening and the second mounting base configured to hold a second portion of the imaging subject protruding from the second lumen opening, wherein the at least a portion of the imaging subject within the lumen is supported between and by the first and second mounting bases; and
a linear stage coupled to the frame between the first and second frame ends, the linear stage configured to translate the at least a portion of the imaging subject along at least a portion of a linear path within the lumen extending between the first and second lumen openings to reposition the imaging subject within the lumen;
wherein the system is further configured to reconstruct a series of 2D photoacoustic images based on the photoacoustic signals detected by the ring ultrasound transducer array at a series of regions of interest obtained by repositioning the imaging subject within the lumen.
